(19) United States
(12) Patent Application Publication
(10) Pub. No.: US 2012/0277637 A1
(43) Pub. Date: Nov. 1, 2012

(54) Method And System For Monitoring Pressure Areas On A Supported Body

(76) Inventors: Alireza Vahdatpour, Redmond, WA (US); Majid Sarrafzadeh, Anaheim Hills, CA (US)

(21) Appl. No.: 13/511,606

(22) PCT Filed: Nov. 17, 2010
(86) PCT No.: PCT/US10/57037

§ 371 Date: May 23, 2012

Related U.S. Application Data
(60) Provisional application No. 61/264,218, filed on Nov. 24, 2009.

(51) Int. Cl. A61B 5/11

(52) U.S. Cl. 600/595

(57) ABSTRACT

A supported body, like the body of a bedridden patient, is monitored by a plurality of pressure sensors located between the body and support surface during an extended period of time. Pressure areas on the body are determined in real time for predetermined time periods during periods of movement between the body and the support surface. Common pressure areas on the body during different body positions on the support surface are determined for predetermined time periods. The pressure areas that exceed a predetermined pressure level for a predetermined time period trigger an alarm indicator and identification of the pressure areas of concern.

### CROSS REFERENCE TO RELATED APPLICATIONS

This application claims the benefit of U.S. Provisional Application Ser. No. 61/264,218 filed Nov. 24, 2009 for Method and Apparatus for Monitoring Bed Condition and Bed User Status to Prevent Pressure Ulcers.

### BACKGROUND OF THE INVENTION

1. Field of the Invention

The present invention relates to a method and system for monitoring pressure areas on a person in a reclining or sitting position, and more specifically relates to monitoring for conditions contributing to the creation of decubitus ulcers.

2. Description of Related Art

The problem of ulcer formation in immobile patients has grown in the United States as the number of patients increased and nursing and support staff, to take care of these patients has decreased. Most serious decubitus ulcers occur in patients that are immobile in hospital beds.

These bedridden patients experience compressive forces and sheer or frictional forces. Compressive forces are caused by pressure on a single point or area of the body, commonly at a weight bearing bony point, of the body such as the hips, heels or elbows. Frictional forces occur when two opposing surfaces slide over and rub against each other, such as when a patient moves about in the bed. It is this combination of forces, both compressive and frictional, that contribute to ulcer formation. Conventional wisdom suggests, that the compressive force component is the single most important factor in ulcer formation.

Compressive and frictional forces exert pressure on an area of the body, which, when sufficiently high, will stop the flow of blood to the pressure affected tissue. The lack of blood to this area of the body deprives the tissue of oxygen. A lack of oxygen causes the tissue to die, forming an ulcer.

It has been found that each patient's tissue tolerance, that is, the pressure threshold which will cause blood to stop flowing to the pressure area, is unique to that individual, and depends on many factors. Whether a certain patient experiences tissue damage is dependent on that patient's specific pressure tolerance.

Today's traditional approach to preventing ulcers in bedridden patients is directed to management of incontinence, nutritional support, and the use of pressure relieving devices such as foam mattresses, gel mattresses, medical grade sheepskins, and alternating pressure devices which continuously adjust pressure levels for at risk regions. The most important practice, however, is frequent patient repositioning. It has been found that repositioning the body reduces or eliminates interface pressure and maintains micro circulation to the at risk regions.

The general practice in the industry is to turn bedridden patients every two to three hours. This practice, however, leaves recurring issues. Some patients may require more frequent rotation, thereby developing decubitus ulcers in spite of the best efforts of the nursing staff.

The limited number of nursing staff and medical support has spurred development of a variety of bed monitoring systems.

For example, U.S. Pat. No. 6,314,451 granted Jan. 1, 2008 for Techniques for Prediction and Monitoring of Clinical Episodes, U.S. Pat. No. 7,077,810 granted Jul. 18, 2006 for Techniques for Prediction and Monitoring of Respiration Manifested Clinical Episodes, and published Application No. 2007/0118054 published May 24, 2007 for Methods and Systems for Monitoring Patients for Clinical Episodes, all revolve around the measuring of recoil movements of the body which result from movement of the heart and blood in the circulatory system. These three documents describe the use of sensors placed at different locations on a mattress pad upon which a patient lies. These sensors are designed to monitor his physiological signs over time. An algorithm predicts or warns against serious clinical episodes such as asthma attacks, shock, myocardio infarction, based on the signals from the sensors.

U.S. Pat. No. 7,825,814 granted Nov. 2, 2010 for a Bed Occupant Monitoring System is directed to a sensor pad on a bed upon which the patient lies, that utilizes optical pressure sensors to provide a quantitative reading for a given number of isolated sensing areas on the patient's body. The patent describes a system by which an alarm can be triggered when the pressure being sensed by the optical pressure sensors exceeds a predetermined threshold.

U.S. Pat. No. 6,485,441 granted Nov. 26, 2002 for a Sensorbed, and U.S. Pat. No. 6,468,234 granted Oct. 22, 2002 for Sleepsmart are directed to a device that monitors a patient's sleep behavior. These patents describe the use of a two layer mattress pad that is able to recognize a patient's body imprint position by using sensors that can collect information such as the patient's position, temperature, and body impulses.

U.S. Pat. No. 6,239,706 granted May 29, 2001 for an In bed State Detection System describes a pressure sensor bed sheet for monitoring whether a patient is present in the bed or not.

U.S. Pat. No. 5,844,488 granted Dec. 1, 1998 for a Bed Sensor and Alarm is directed to a pressure sensitive sensor pad that has a central pressure sensor to determine the presence of a patient in the center of a bed and additional sensors located at the edge of the bed to detect patient movement towards either edge of the bed. This patent, like U.S. Pat. No. 6,239,706, is mainly concerned with providing an early warning signal when a patient appears to be exiting the bed without assistance.

In spite of these and many other bed monitoring systems, the recurring issues contributing to the formation of decubitus ulcers on immobile patients have not been adequately addressed, let alone solved. These systems do not adequately monitor at risk areas for each patient. Nor do they provide efficient reminders to reposition the patient. Nor are they adjustable, taking into consideration the patient's individual pressure tolerances.

The present invention addresses and moves a long way towards providing a system that will prevent decubitus ulcer formation by identifying pressure areas on a human body, measuring pressure levels in real time, providing position tracking of the patient on a support surface, and providing a system that can be adjusted for each individual patient.

### SUMMARY OF THE INVENTION

The method and system for monitoring pressure areas on a supported body, in real time, throughout periods of movement between the body and a support surface of the present invention is an improvement. Each position of a body on the support surface generates a pressure map of the body in that position. The pressure map indicates location of pressure areas, pressure level for each pressure area on the body, and duration of a pressure level in area. Each time the body changes position on the support surface, a new pressure map is generated. Common pressure areas between the previous and new pressure map is determined. The pressure level for each common area is monitored to determine if the pressure level exceeds a predetermined level for a predetermined time frame. The pressure limit and time limit are unique and are adjusted accordingly.

### BRIEF DESCRIPTION OF THE DRAWINGS

The exact nature of this invention, as well as the objects and advantages thereof, will become readily apparent from consideration of the following specification in conjunction with the accompanying drawings in which like reference numerals designate like parts throughout the figures thereof and wherein:

FIG. 1 is a pictorial and block diagram illustration of a monitoring system according to an embodiment of the present invention;

FIG. 2 is a pictorial representation of an array of pressure sensors formed in a sheet that may be used in the system of the present invention;

FIG. 3 is a graphical illustration of a textile pressure sensor that may be used in the system of the present invention;

FIG. 4 is a graphical illustration of a pressure map generated by the present invention, showing a patient on his back;

FIG. 5 is a graphical illustration of a pressure map generated by the present invention, showing a patient on his side;

FIG. 6 is a graphical illustration of the present invention showing a patient on his back;

FIG. 7 is a graphical illustration of the conjectured position of a patients body parts and significant pressure points in the pressure map of FIG. 6;

FIG. 8 is a graphical illustration of a pressure map generated by the present invention of a patient lying on his side;

FIG. 9 is a graphical illustration of the conjectured position of a patients body parts significant pressure points of the pressure map of FIG. 8;

FIG. 10 is a graphical illustration of five significant pressure areas of a patient lying on his side;

FIG. 11 is a graphical illustration of five significant pressure areas of the same patient lying on his side after some body movement;

FIG. 12 is a graphical illustration of a pressure map of a patient on his back;

FIG. 13 is a graphical illustration of a pressure map generated by the present invention after the patient, of FIG. 12, moved by rotating around the vertical axis;

FIG. 14 is a graphical illustration of a pressure map generated by the present invention after the patient of FIG. 12 moved laterally;

FIG. 15 is a graphical illustration of a pressure maps of a patient on his back;

FIG. 16 is a graphical illustration of a pressure map of the patient of FIG. 15 lying on his side;

FIG. 17 is a graphical illustration of common pressure areas between the two patient positions of FIGS. 15 and 16; and

FIG. 18 is a diagrammatic flow of a preferred method of the present invention.

### DETAILED DESCRIPTION OF THE PREFERRED EMBODIMENTS

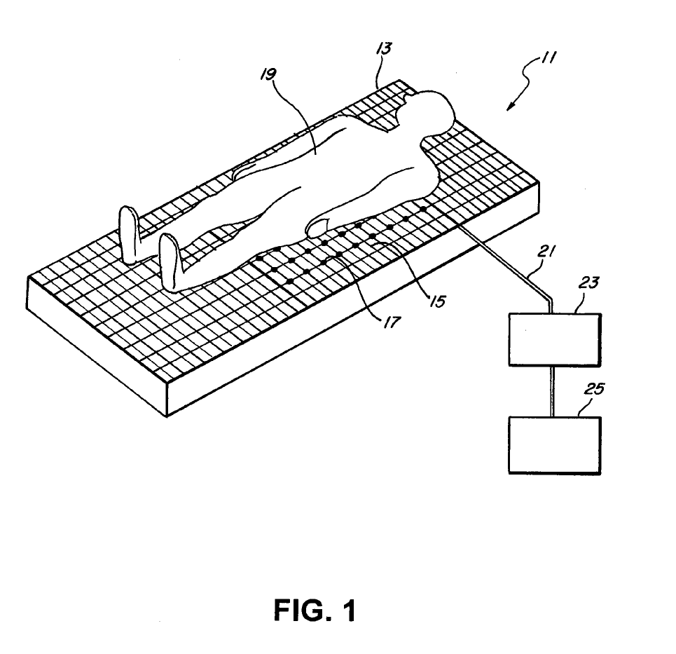

FIG. 1 illustrates a system 11 for monitoring pressure areas on a supported body 19, reclining on a support surface 13. Support surface 13 has a matrix 15 of sensors 17 extending across the entire support surface 13, which may be a bed, for example. The invention is equally applicable to a support surface for a body in a sitting position, such as in a wheelchair, for example. In a wheelchair the matrix of sensors (not shown) would be covering the seat of the wheelchair. Although the preferred embodiment of the present embodiment is illustrated with a human body 19 in a reclining position, it should not be so limited.

The matrix of sensors 15 are configured to communicate by communication line 21, which may be any well known communication link, including wireless, with a processor and memory configuration 23 that receives all the signals from each of the sensors 17 in the matrix of sensors 15. The processor 23 performs storing and analysis functions, as will be fully described hereinafter. The processor determines whether there are certain pressure areas on the body 19 which exceed a predetermined limit for a predetermined time. Upon such a determination, the processor 23 generates a signal such as an alarm or notice, to a station 25 which may, for example, be a nurse monitoring station. Besides an alarm, information about the location of the particular pressure area on the body that has caused the alarm, is provided.

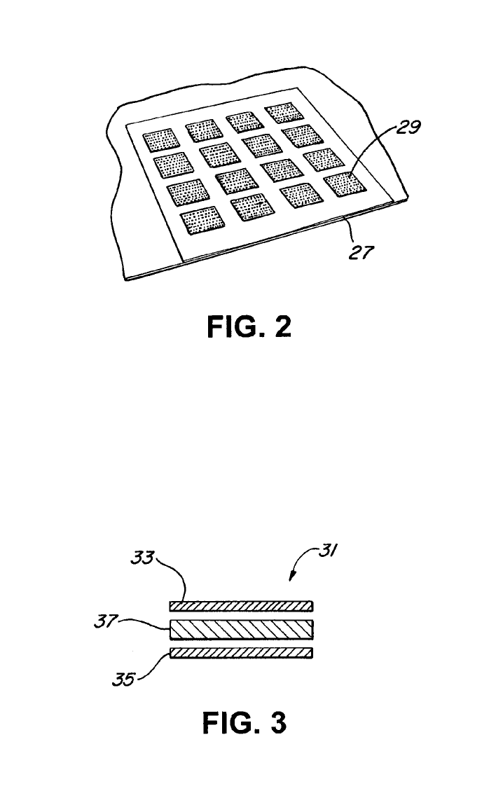

FIG. 2 is an illustration of a sensor matrix having a plurality of sensors 29 formed into a bed sheet 27. The sensor should go under a patient in a reclining position, as shown in FIG. 1, or on a seat for a seated patient. The size of the sheet 27 is sized to accommodate either use.

An alternate and preferred pressure matrix especially suited for use on a bed is the use of a resistive textile sensor 31 as shown in FIG. 3. The construction of the textile pressure sensor 31 provides the same touch and feel as normal fabric. The sheet is constructed in a three layer stack. The top layer 33 and the bottom layer 35 are made with the same type of conductive fabric, Type I for example. This Type I material does not respond to normal pressure or tension by changing the characteristics of the fabric. The middle layer 37 is another type of fabric, Type II for example. This Type II fabric is sensitive to the external forces applied to it. It has characteristics that change in response to these forces. Force would change the characteristics of the middle layer 37, such as resistivity or capacitance, for example, allowing that change to be measured through the top and bottom layer. These changes to the characteristics of the middle layer 37 are then used to calculate the external force being sensed by sensor 31.

Regardless of the type of pressure sensors or pressure sensors or pressure sensor arrangement being utilized, each pressure sensor is connected to the processor 23 which stores the location of each of the sensors in the pressure matrix 15, and the pressure being sensed by each sensor, in real time.

The use of the pressure sensor 31 of FIG. 3 is of considerable advantage over other pressure sensors 29, which are independently formed into a sheet 27. A much larger amount of sensors can be located in a matrix using sensor construction 31.

The processor 23 may be a programmed general purpose computer or a special firmware device dedicated to the information collection and analysis required for this invention.

The following discussion with respect to the function of the preferred embodiment of the invention will be directed to a body being supported on a bed. It should be remembered, however, that the principles and functions being described are equally applicable to the support of only portions of a body, such as when a body is in a seated position.

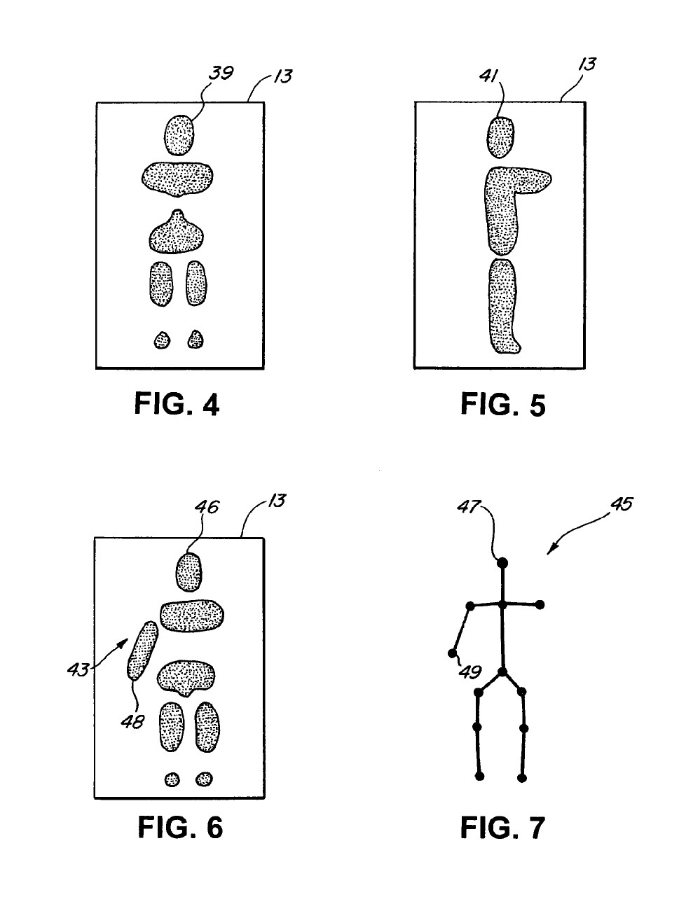

FIG. 4 illustrates a pressure pattern 39 of a certain individual lying on his back on a support surface 13. This pressure pattern 39 is unique to each individual and will vary depending upon the individual's size, weight, shape, etc. The pressure pattern 39 is essentially a pressure map of that individual, lying on his back on the support surface 13.

FIG. 5 illustrates a pressure map 41 generated for the same individual lying on his left side on the support surface 13. The change in pressure patterns, which causes two completely different pressure maps 39 and 41, is an indication of movement of the individual from his back to his side. Such movement can be tracked by assigning degrees to each position. The flat on the back position of FIG. 4 could be assigned 0 degrees. The left side position could be assigned 90 degrees.

Tracking movement of a body on the support surface over real time is an important feature of the present invention.

FIG. 6 illustrates a pressure map 43 of an individual on his back. The pressure map has various pressure areas like pressure area 46 around his head, and pressure area 48 around his hand.

In order to help track movement of a individual on the support surface 13, the pressure map information is distilled down to a diagram of connected body parts 45 (FIG. 7). The diagram 45 essentially tracks the pressure areas, like pressure area 47 for the head and pressure area 49 for the hand, for all the body parts, but in a representative manner, with much less data.

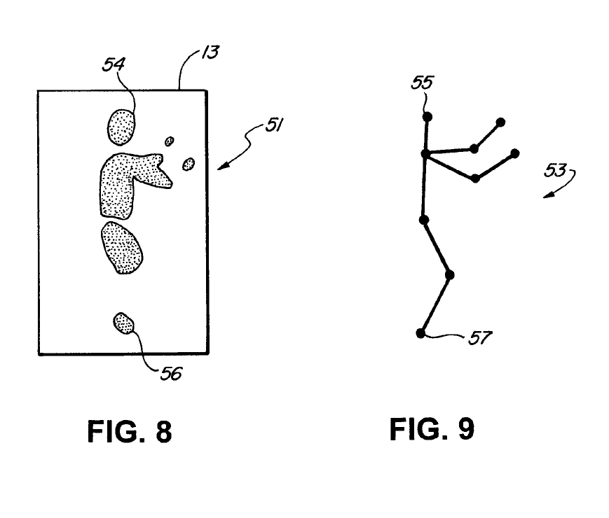

FIG. 8 illustrates a pressure map 51 of a body lying on a support surface 13 on his left side. In other words, at 90°. The pressure map has pressure areas, like pressure area 54 at his head and pressure area 56 at his feet.

FIG. 9 illustrates a connected body part diagram 53 that is distilled from the pressure map 51 of FIG. 8. Body part diagram 53 tracks the various pressure areas, like pressure area 55 at the head and pressure area 57 at the feet, of the pressure map 51.

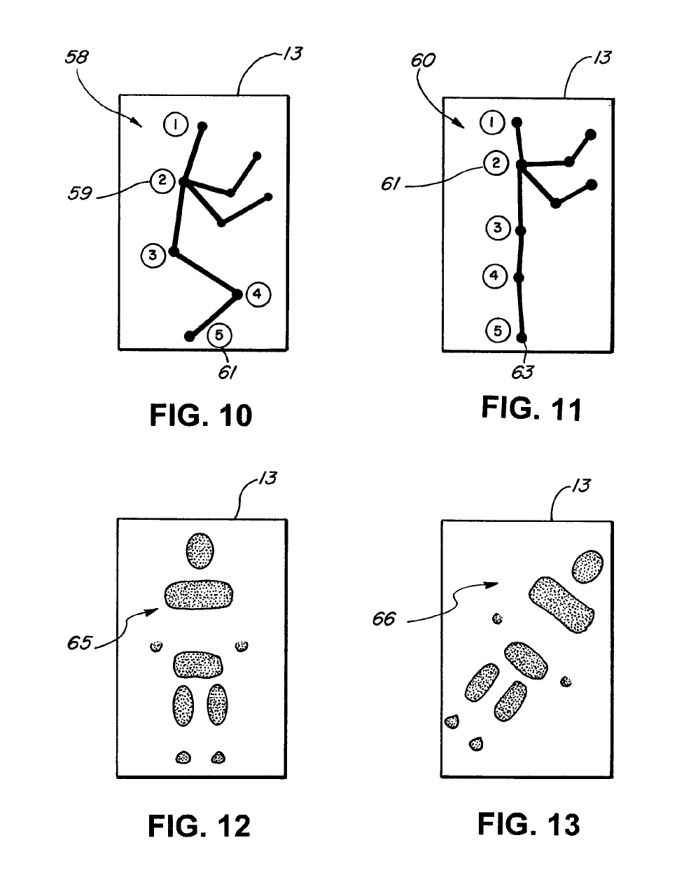

The diagrams of connected body parts are used to quickly determine movement of the body on a support surface 13, as illustrated in FIGS. 10 and 11. The diagram of connected body parts 58 of FIG. 10 illustrates five distinct pressure areas and shows that the body is in the 90° position, on the left side. Location of pressure areas, such as the shoulder area, number 2 position 59 and the feet area number 5 position 61, for example, are clearly tracked.

FIG. 11 shows the body of FIG. 10 laying on support surface 13 on the left side with some of the body parts moved. The legs which were previously bent are now straight. The connected body parts diagram 60 of FIG. 11 shows the foot pressure area, number 5 position 63 the shoulder pressure area number 2 position 61 also have moved, as have parts of the arms.

Tracking movement of a body on a support surface 13 is important. But, the present invention goes further by detecting the common areas of pressure between the two positions of the body, such as shown in FIGS. 10 and 11, for example.

The common areas of pressure between one position and another position of the body is determined by applying a series of image matching techniques, such as registration and warping. The mathematical process of matching common pressure areas from two different pressure maps utilizing is well known and discussed in “Registration of Translated and Rotated Images Using Finite Fourier Transforms” by E. De Castro and C. Mirandi in Pattern Analysis and Machine Intelligence, IEEE Transactions, Vol. PAMI-9, No. 5, pages 700-703, September 1987. Another article relating to the same subject is “A Class of Algorithms for Fast Digital Registration” by D. I. Barnea and H. F. Smverdman, 1972, IEEE Transactions, Computers, C-21, pages 179-187. These articles are fully incorporated herein. The mathematical and algorithmic aspects of image warping are discussed in “Image warping Using Few Anchor Points and Radial Functions” by N. Arad and D. Reisfeld in Computer Graphics Forum, Vol. 4, No. 1, 1995, pages 35-46. Another article is by Paul S. Heckbert. “Fundamentals of Texture Mapping and Image Warping”. Technical Report. University of California at Berkeley, Berkeley, Calif., USA. These articles are fully incorporated herein.

By using registration and warping techniques on the pressure map data generated by different pressure maps created as a result of the body's movement on the support surface 13, the common pressure areas between the two positions can be determined.

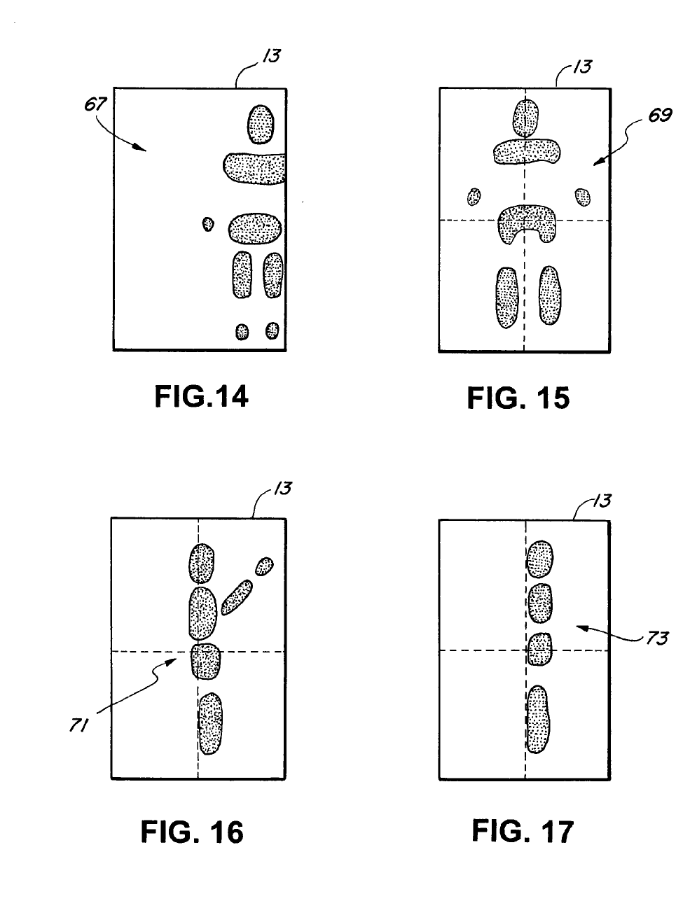

FIG. 12 shows a pressure map 65 generated by a body laying flat, at 0 degrees on a support surface 13. If the body rotates around the vertical axis, a pressure map 66 (FIG. 13) is generated. If the body translates from the middle of the bed (FIG. 12) to the side of the bed (FIG. 14), pressure map 67 is generated.

In both situations, the system of the present invention determines the common pressure areas between pressure maps 65 and 66 and 65 and 67, or pressure maps 66 and 67.

An example of determining the common pressure areas between two pressure maps is illustrated in FIGS. 15, 16 and 17.

FIG. 15 shows a pressure map 69 of a body lying flat at 0 degrees on a support surface 13.

FIG. 16 shows a pressure map 71 of that same body lying on its left side on support surface 13.

In order to determine the common pressure areas between these two pressure maps 69 and 71, a registration technique is applied to align the relative position of the supported body in the two images. This will compensate for any lateral movement or rotation around a vertical axis. Next, a warping technique is applied to the extracted body diagrams to match body parts between the two pressure maps. Finally, body areas that are common between the two pressure maps are determined (as in map 73).

It is the common pressure area of map 73 which are of most concern and interest. When a body is moving around on the support surface 13, intuitively one would conclude that a condition giving rise to decubitus ulcers is not present. The fact that common pressure areas 73 exist between two different pressure maps, representing two distinctly different body positions, indicates that decubitus ulcer formation is still a problem.

Tracking movement of a body on a support surface and tracking these common pressure areas, over real time, provide a more confident indication of conditions on the body that can lead to decubitus ulcer formation. If the common pressure areas 73 exceeds the predetermined pressure level or a predetermined limit, for longer than a predetermined time, a condition of the body that can lead to decubitus ulcer formation is identified.

The pressure limit and time limit is preferably determined by medical staff according to the particular sensitivities of the body being monitored.

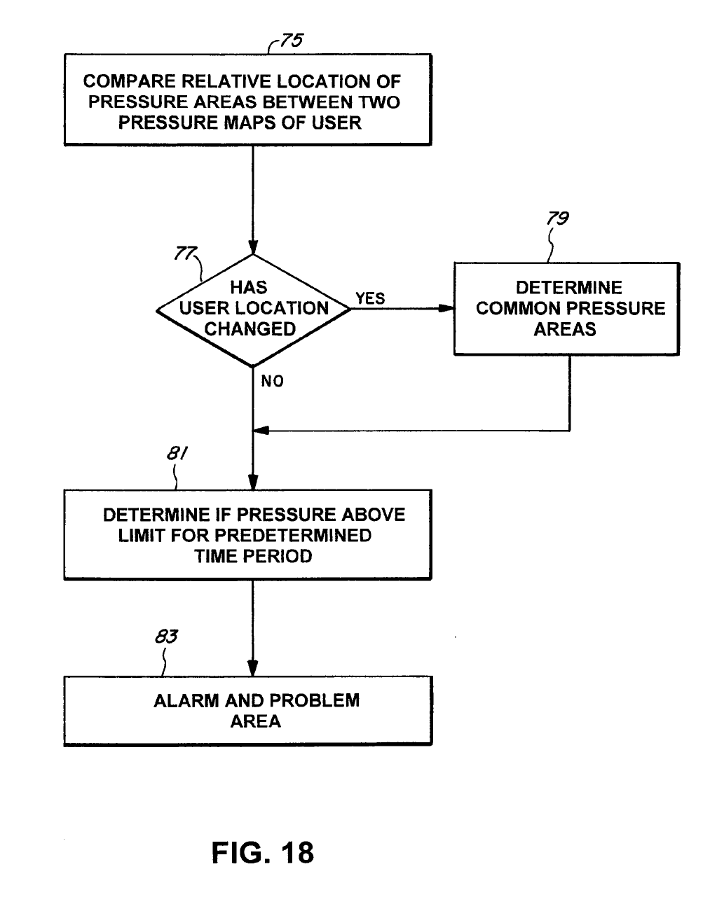

The processor determines if the pressure upon any area of the body exceeds a pressure limit for a predetermined time period, throughout movement of the body on a support surface, according to the procedure illustrated in FIG. 18.

The processor receives all of the pressure map data and generates diagrams of connected body parts in order to compare the relative location of pressure areas designated in one time frame to the relative location of the same pressure areas in a following time period (75). If the like pressure areas are located in different locations, this is an indication that the body has changed position (71). A determination of common pressure areas (79) is then made, in the manner described above. Once these common pressure areas are identified, it is determined if the pressure in these common pressure areas is above a predetermined limit for a predetermined time period (81). If these common pressure areas are above a predetermined limit for a predetermined time period, the processor will send out an alarm (83) along with an indication of the location of the pressure areas which are considered to be problematic.

If a comparison between the two diagram indicates that the body has not changed location (77) because the two pressure areas in the two diagrams are in the same location, a determination must still be made if these pressure areas are above a predetermined limit for a predetermined time (81). As might be anticipated, a body that is not moving, has a greater chance of exhibiting higher pressure over a longer period of time. If the limits are exceeded, an alarm (83) is triggered along with an indication of the pressure areas of concern.

What is claimed is:

1. A method for monitoring a supported body for pressure areas, the steps of the method comprising:
continuously sensing pressure areas and pressure limits on the body in a first position on a support surface;
continuously sensing pressure areas and pressure levels on the body in a second position on the support surface;
determining common pressure areas sensed in the first position and the second position; and
determining whether the pressure level sensed for the common pressure areas exceeds a predetermined limit for a predetermined time.

2. The method of claim 1 further comprising generating an alert when the pressure sensed exceeds a predetermined level for a predetermined period of time.

3. The method of claim 1 wherein the first position and second position are the same.

4. The method of claim 1 wherein the determining step occurs after a change in body position in the support surface.

5. The method of claim 1 wherein continuously sensing pressure areas and pressure levels comprises generating a pressure map updated in real time.

6. The method of claim 1 wherein common pressure areas between the first position and second position are determined by a registration technique.

7. A method for monitoring a supported body for pressure areas, the steps of the method comprising:
continuously sensing pressure areas and pressure levels on the body for a first predetermined time period;
generating a first pressure map from the pressure areas in the first time period;
continuously sensing pressure areas and pressure levels on the body for a second predetermined time period;
generating a second pressure map from the pressure areas sensed in the second time period;
comparing the first and second pressure maps for similarity in size and shape; and
if first and second pressure maps are the same, indicating that the body has not moved, determining if any pressure area exceed a predetermined pressure limit for a predetermined time period.

8. The method of claim 7 further comprising:
if the first and second pressure maps are not the same, indicating that the body has moved, determining the common pressure areas in the first and second pressure maps; and
determining if any common pressure areas pressure limit for a predetermined time period.

9. An apparatus for monitoring a supported body for pressure areas, comprising:
a support platform for supporting at least a part of a human body;
a network of pressure sensors covering the support platform; and
a processor and computer readable storage connected to the network of pressure sensors for continuously receiving pressure signals from the pressure sensors, the processor generating a first pressure map based on the pressure signals received for a first predetermined time period and a second pressure map based on the pressure signals received for a second predetermined time period, the processor comparing the first and second pressure maps for similarity in size and shape.

10. The apparatus of claim 9 wherein the processor compares the first and second pressure maps and determines that the body has not moved with respect to the support platform if the first and second pressure maps are the same in size and shape.

11. The apparatus of claim 9 wherein the processor compares the first and second pressure maps and determines that the body has moved with respect to the support platform if the first and second pressure maps are not the same in size and shape.

12. The apparatus of claim 10 wherein the processor determines if any pressure area in the first and second pressure maps exceeded a predetermined pressure limit for a predetermined time period.

13. The apparatus of claim 11 wherein the processor determines common pressure areas in the first and second pressure maps and for the common pressure areas determines if any exceeded a predetermined pressure limit for a predetermined time period.

14. The apparatus of claim 9 wherein the support platform is a bed for supporting the entire body and the network of pressure sensors are individual sensors located in a sheet covering the bed.

15. the apparatus of claim 9 wherein the support platform is a bed for supporting the entire body and the method of pressure sensors is a sheet for covering the bed that has multiple conductive layers adapted to sense pressure.
